(19) United States
(12) Patent Application Publication
(10) Pub. No.: US 2020/0402648 A1
(43) Pub. Date: Dec. 24, 2020

(54) GENERATING HIGH CONFIDENCE REFILLS FOR UNIFIED WORKFORCE MANAGEMENT

(71) Applicant: Walmart Apollo, LLC, Bantonville, AR (US)

(72) Inventors: Shreyan Ghosh, West Bengal (IN); Akash Verma, Bengaluru (IN); Prateek Mishra, Bangalore (IN)

(21) Appl. No.: 16/531,291

(22) Filed: Aug. 5, 2019

(30) Foreign Application Priority Data
Jun. 24, 2019 (IN) 201941025044

(51) Int. Cl. G16H 40/20; G16H 20/10

(52) U.S. Cl. CPC G16H 40/20; G16H 20/10

(57) ABSTRACT

Examples provide a workforce manager that analyzes historical pharmacy transaction data to generate a set of forecasted future prescriptions for a selected pharmacy. The forecasted future prescriptions are divided into predicted new prescriptions and predicted refill prescriptions. The predicted refill prescriptions are classified into a set of high confidence refills and a set of low confidence refills. The labor demand associated with the set of high confidence refills are redistributed within a range of pickup dates associated with each high confidence refill prescription to smooth labor demand minimizing variation within a selected time-period. A number of personnel are identified for each day in the selected time-period based on the smoothed labor demand. A schedule is published assigning at least a portion of the forecasted future prescriptions and the number of personnel to each day in the selected time-period is output.

### BACKGROUND

Determining the correct number of staff to adequately process all incoming prescriptions and orders for a pharmacy or other store is a difficult and frequently inaccurate task. Currently, staffing decisions are made based on previous numbers of prescriptions coming through the pharmacy. However, this frequently results in overstaffing or understaffing. This is an inefficient process leading to suboptimal workforce allocations.

### SUMMARY

Some examples provide a system for performing unified pharmacy workforce management. The system includes at least one processor communicatively coupled to a memory. A machine learning component analyzes per-store historical pharmacy transaction data to generate forecasted future prescriptions. The forecasted future prescriptions comprising a set of predicted refill prescriptions and a set of predicted new prescriptions at a per-day level. A forecast component calculates predicted labor demand on the per-day level based on the forecasted future prescriptions and estimated per-script processing time data. A refills confidence component separates the set of predicted refill prescriptions into a set of high confidence refills and a set of low confidence refills based on historical refill data and medication data associated with each refill prescription in the set of predicted refill prescriptions. A smoothing component reassigns labor demand associated with processing refill prescriptions in the set of high confidence refills to an earlier date or a later date within a range of possible pickup dates associated with a refill due date for each refill prescription in the set of high confidence refills to minimize labor demand variation across a set of days. An assignment component calculates a number of personnel sufficient to meet the predicted labor demand for each day in the set of days. An output device outputs a schedule assigning the calculated number of personnel to each workstation associated with the smoothed labor demand for each day in the set of days.

Other examples provide a computer-implemented method for performing unified pharmacy workforce management. A forecast component calculates labor demand on a per-day level for a selected pharmacy based on a set of forecasted future prescription refills and estimated per-script processing time for each prescription in the set of forecasted future prescriptions. A refills confidence component generates a set of high confidence refills and a set of low confidence refills based on historical refill data and medication data associated with each refill prescription in the set of predicted refill prescriptions. A smoothing component moves labor demand associated with processing refill prescriptions in the set of high confidence refills to an earlier date or a later date within a range of possible refill dates associated with a refill due date for each prescription in the set of high confidence refills to smooth labor demand across a set of days. An assignment component calculates a number of personnel to meet the predicted labor demand for each day in the set of days. A scheduling component outputs a schedule assigning the calculated number of personnel to each day in the set of days to meet the predicted labor demand.

Still other examples provide a computer storage device, having computer-executable instructions for performing unified pharmacy workforce management by a workforce manager component. The workforce manager component analyzes per-store historical pharmacy transaction data; generates forecasted future prescriptions based on the analysis results, the forecasted future prescriptions comprising a set of predicted refill prescriptions and a set of predicted new prescriptions at a per-day level; calculates labor demand on the per-day level based on the forecasted future prescriptions and estimated per-script processing time data; creates a set of high confidence refills and a set of low confidence refills based on historical refill data and medication data associated with each refill prescription in the set of predicted refill prescriptions; smooths labor demand across a set of days by moving labor demand associated with processing at least one refill prescription in the set of high confidence refills to an earlier date or a later date within a range of predicted refill dates for each high confidence refill; and outputs a number of personnel associated with the smoothed labor demand for utilization in creating a workforce schedule for each day in the set of days.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an exemplary block diagram illustrating a system for pharmacy workforce management based on forecast future prescriptions.

FIG. 2 is an exemplary block diagram illustrating a workforce manager component.

FIG. 3 is an exemplary block diagram illustrating a set of forecasted future prescriptions.

FIG. 4 is an exemplary block diagram illustrating a workforce manager component forecasting customer demand at a per-day level.

FIG. 5 is an exemplary block diagram illustrating a workforce manager component estimating raw labor demand at a store-day level.

FIG. 6 is an exemplary block diagram illustrating a prediction component generating predicted pickup dates for forecasted refill prescriptions.

FIG. 7 is an exemplary block diagram illustrating a smoothing component smoothing labor demand across a set of days.

FIG. 8 is an exemplary block diagram illustrating a database storing historical pharmacy transaction data for pharmacy workforce management.

FIG. 9 is an exemplary flow chart illustrating operation of the computing device to output a schedule assigning personnel to pharmacy workstations based on predicted labor demand associated with forecasted future prescriptions.

FIG. 10 is an exemplary flow chart illustrating operation of the computing device to analyze historical data at a per-pharmacy level to perform personnel scheduling based on high confidence refills.

FIG. 11 is an exemplary flow chart illustrating operation of the computing device to update a workforce schedule based on total forecast prescriptions.

FIG. 12 is an exemplary bar graph illustrating total predicted labor demand across a set of days.

FIG. 13 is an exemplary bar graph illustrating smoothed labor demand across a set of days.

FIG. 14 is an exemplary line graph illustrating smoothed labor demand across a set of days.

Corresponding reference characters indicate corresponding parts throughout the drawings.

### DETAILED DESCRIPTION

A more detailed understanding can be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that can in isolation and out of context be read as absolute and therefore limiting, can only properly be read as being constructively preceded by a clause such as “In at least some examples, . . . ” For brevity and clarity of presentation, this implied leading clause is not repeated ad nauseum.

Referring to the figures, examples of the disclosure enable workforce management at a pharmacy based on forecasted future prescriptions. In some examples, a workforce manager component determines the number of personnel to assign to each workstation at a pharmacy based on predicted new-fill prescriptions and predicted refill prescriptions. A new fill prescription is a new prescription brought randomly into a pharmacy. A refill prescription is a recurring prescription which is refilled on a regular or semi-regular basis. The system enables more efficient allocation of labor demand across a set of days for optimal resource allocation, reduction in personnel idle time and prevention of overstaffing or understaffing.

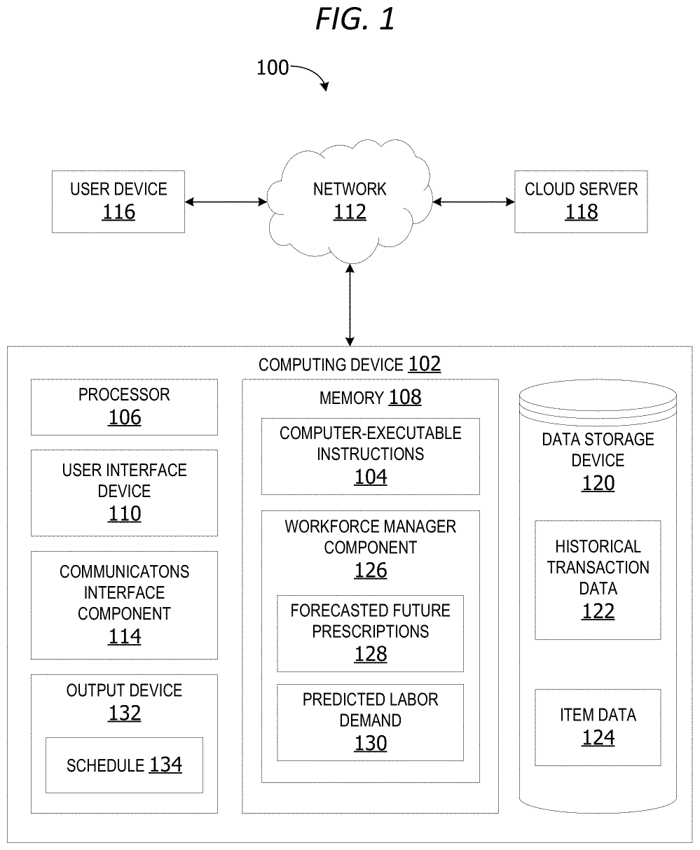

Referring again to FIG. 1, an exemplary block diagram illustrates a system 100 for pharmacy workforce management based on forecast future prescriptions. In the example of FIG. 1, the computing device 102 represents any device executing computer-executable instructions 104 (e.g., as application programs, operating system functionality, or both) to implement the operations and functionality associated with the computing device 102. The computing device 102 in some examples includes a mobile computing device or any other portable device. A mobile computing device includes, for example but without limitation, a mobile telephone, laptop, tablet, computing pad, netbook, gaming device, and/or portable media player. The computing device 102 can also include less-portable devices such as servers, desktop personal computers, kiosks, or tabletop devices. Additionally, the computing device 102 can represent a group of processing units or other computing devices.

In some examples, the computing device 102 has at least one processor 106 and a memory 108. The computing device 102 in other examples includes a user interface device 110.

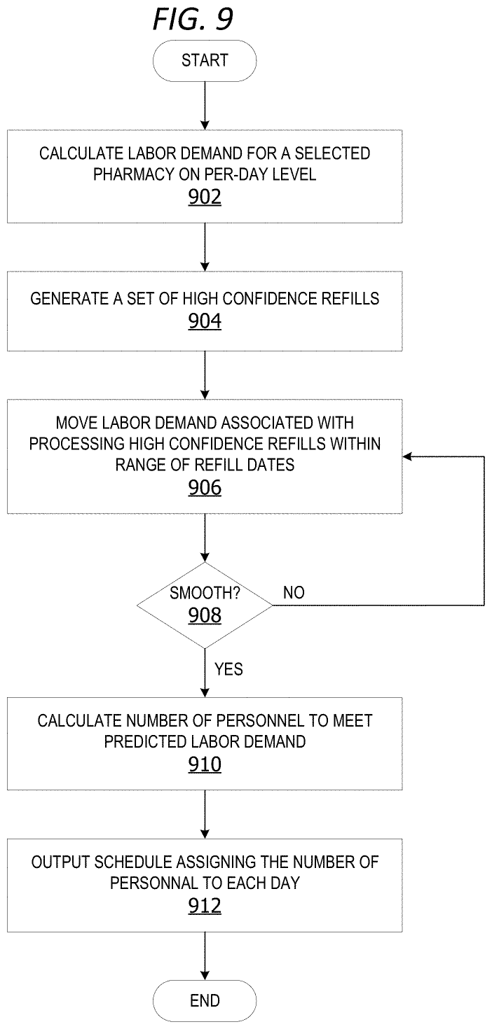

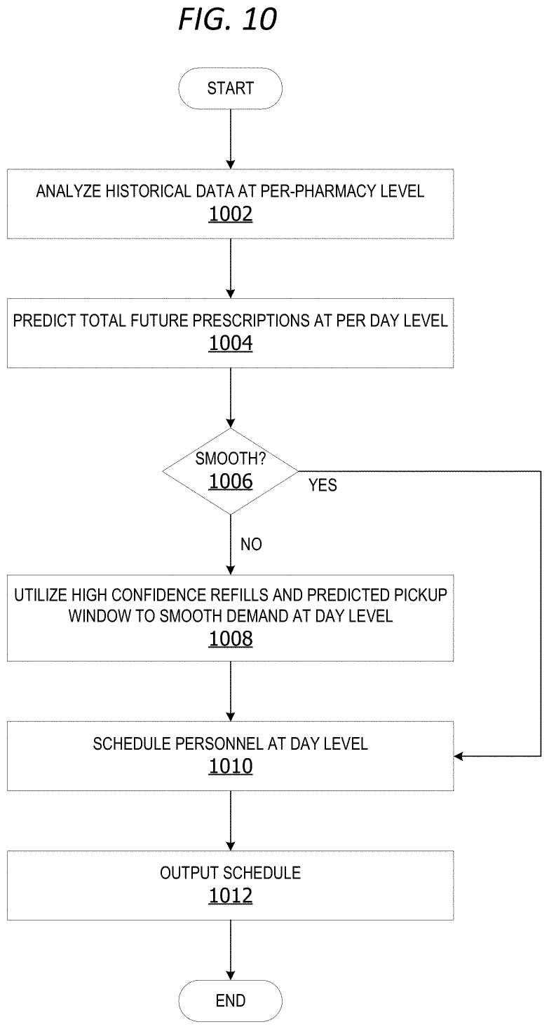

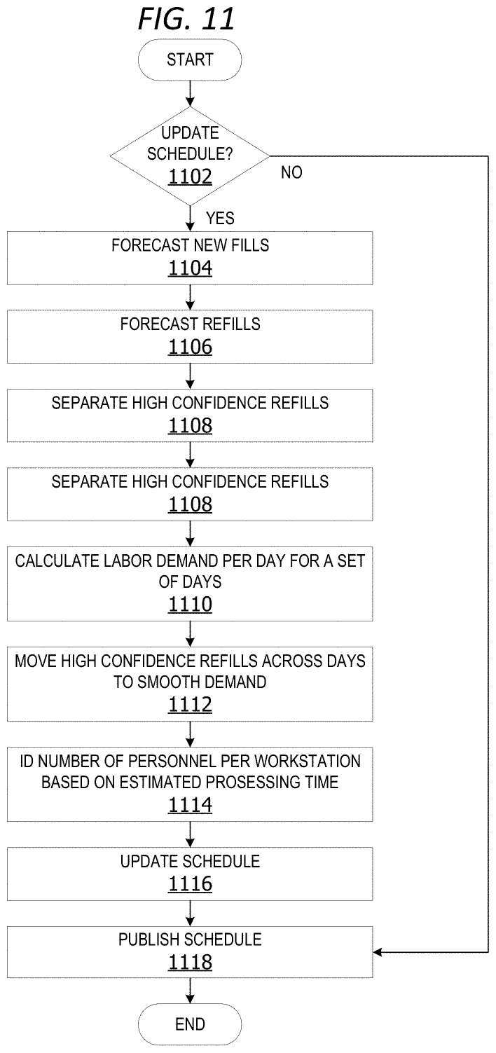

The processor 106 includes any quantity of processing units and is programmed to execute the computer-executable instructions 104. The computer-executable instructions 104 is performed by the processor 106, performed by multiple processors within the computing device 102 or performed by a processor external to the computing device 102. In some examples, the processor 106 is programmed to execute instructions such as those illustrated in the figures (e.g., FIG. 9, FIG. 10 and FIG. 11).

The computing device 102 further has one or more computer-readable media such as the memory 108. The memory 108 includes any quantity of media associated with or accessible by the computing device 102. The memory 108 in these examples is internal to the computing device 102 (as shown in FIG. 1). In other examples, the memory 108 is external to the computing device (not shown) or both (not shown). The memory 108 can include read-only memory and/or memory wired into an analog computing device.

The memory 108 stores data, such as one or more applications. The applications, when executed by the processor 106, operate to perform functionality on the computing device 102. The applications can communicate with counterpart applications or services such as web services accessible via a network 112. In an example, the applications represent downloaded client-side applications that correspond to server-side services executing in a cloud.

In other examples, the user interface device 110 includes a graphics card for displaying data to the user and receiving data from the user. The user interface device 110 can also include computer-executable instructions (e.g., a driver) for operating the graphics card. Further, the user interface device 110 can include a display (e.g., a touch screen display or natural user interface) and/or computer-executable instructions (e.g., a driver) for operating the display. The user interface device 110 can also include one or more of the following to provide data to the user or receive data from the user: speakers, a sound card, a camera, a microphone, a vibration motor, one or more accelerometers, a BLUETOOTH® brand communication module, global positioning system (GPS) hardware, and a photoreceptive light sensor. In a non-limiting example, the user inputs commands or manipulates data by moving the computing device 102 in one or more ways.

The network 112 is implemented by one or more physical network components, such as, but without limitation, routers, switches, network interface cards (NICs), and other network devices. The network 112 is any type of network for enabling communications with remote computing devices, such as, but not limited to, a local area network (LAN), a subnet, a wide area network (WAN), a wireless (Wi-Fi) network, or any other type of network. In this example, the network 112 is a WAN, such as the Internet. However, in other examples, the network 112 is a local or private LAN.

In some examples, the system 100 optionally includes a communications interface component 114. The communications interface component 114 includes a network interface card and/or computer-executable instructions (e.g., a driver) for operating the network interface card. Communication between the computing device 102 and other devices, such as but not limited to, a user device 116 and/or a cloud server 118, can occur using any protocol or mechanism over any wired or wireless connection. In some examples, the communications interface component 114 is operable with short range communication technologies such as by using near-field communication (NFC) tags.

The user device 116 represent any device executing computer-executable instructions. The user device 116 can be implemented as a mobile computing device, such as, but not limited to, a wearable computing device, a mobile telephone, laptop, tablet, computing pad, netbook, gaming device, and/or any other portable device. The user device 116 includes at least one processor and a memory. The user device 116 can also include a user interface device.

The cloud server 118 is a logical server providing services to the computing device 102 or other clients, such as, but not limited to, the user device 116. The cloud server 118 is hosted and/or delivered via the network 112. In some non-limiting examples, the cloud server 1xx is associated with one or more physical servers in one or more data centers. In other examples, the cloud server 118 is associated with a distributed network of servers.

The system 100 can optionally include a data storage device 120 for storing data, such as, but not limited to historical transaction data 122 and/or item data 124. The historical transaction data 122 is data associated with previous/historical prescriptions, including both new prescriptions and refill prescriptions at a selected store or pharmacy location. The historical transaction data 122 can include prescription pickup date (date of refill), type of medication, number of previous refills used/picked up for that medication in the past, how close to refill due date prescription was picked up, etc.

The item data 124 is data associated with a prescription. The item data 124 can include, without limitation, the type of medication, name of the medication, number of refills for the medication, etc.

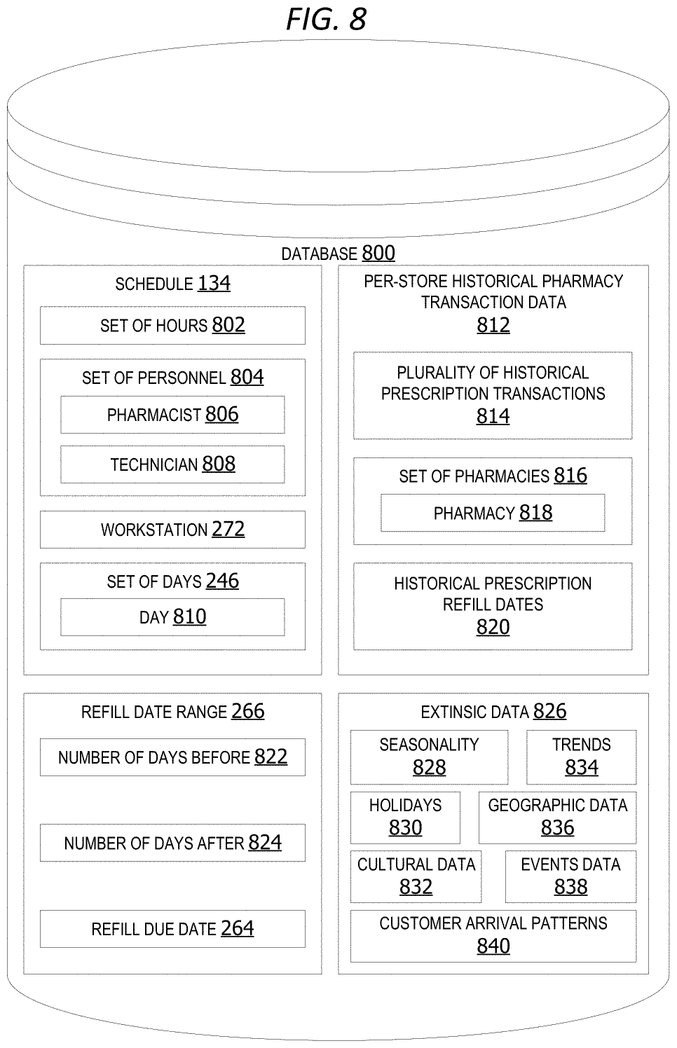

The data storage device 120 can include one or more different types of data storage devices, such as, for example, one or more rotating disks drives, one or more solid state drives (SSDs), and/or any other type of data storage device. The data storage device 1xx in some non-limiting examples includes a redundant array of independent disks (RAID) array. In other examples, the data storage device 120 includes a database such as, but not limited to, the database 800 in FIG. 8.

The data storage device 120 in this example is included within the computing device 102, attached to the computing device, plugged into the computing device or otherwise associated with the computing device 102. In other examples, the data storage device 120 includes a remote data storage accessed by the computing device via the network 112, such as a remote data storage device, a data storage in a remote data center, or a cloud storage.

The memory 108 in some examples stores one or more computer-executable components, such as, but not limited to, the workforce manager component 126. The workforce manager component 126 analyzes historical transaction data 122 and/or item data 124 to generate forecasted future prescriptions 128 which are likely to be brought in/filled at the pharmacy within a given set of future days (future time-period). The workforce manager component 126 generates a predicted labor demand 130 used to determine the number of people needed at each workstation in the pharmacy on each day in the future time-period based on the number of forecasted future prescriptions and estimated processing time to fill each type of prescription in the forecasted future prescriptions. The workforce manager component smooths the predicted labor demand using high confidence refill data to minimize labor demand across each day in the set of days within the future time-period.

In some examples, the workforce manager component 126 generates a schedule 134 assigning a number of personnel (pharmacists and/or technicians) to each workstation in the pharmacy for each day in the set of days based on the smoothed labor demand. The schedule 134 is output to a user via an output device 132. The output device 132 is any type of device for outputting data, including, without limitation, a display screen, a speaker, a printer, a projector, a touchscreen, or any other device. In other examples, the schedule 134 is output via the user interface device 110.

The schedule in still other examples, can be transmitted to a user device 116 via the network 112 for viewing by one or more users associated with the user device 116. The schedule 134 in still other examples, is saved into a database associated with the data storage device 120.

In this example, the computing device 102 sends and receives data from the user device 116 and/or the cloud server 118. In other examples, the computing device operates in an absence of a network.

Likewise, in this example, the workforce manager component obtains historical transaction data and/or item data from the data storage device 120 on the computing device. In other non-limiting examples, the workforce manager component obtains the historical transaction data and/or item data from the cloud server or other cloud storage device via the network 112.

The workforce manager component improves labor forecast irregularities, which are caused whenever labor hours are determined based on prescription demand alone. The system is useful in labor staffing and optimal distribution of labor thereby creating days with more or less equal workload. The system enables improved forecast of labor demand on a per-day basis for better optimization of labor resources. Existing labor resources can be moved to new stores, from which large labor demand is expected. The system enables more accurate reduction in overestimation due to unaccounted labor hours will lead to savings through reduced labor hour allocations. The system can also be extended to estimate labor hours caused due to opening of a new store and remodel of a store.

In other examples, the system takes into account geospatial exploration in terms of urban density, climate, and cultural hotspots, income can be incorporated to determine optimal customer arrival probabilities. The system calculates labor hours from script demand through use of variables like type of prescription, processing time for each prescription etc. for improved scheduling.

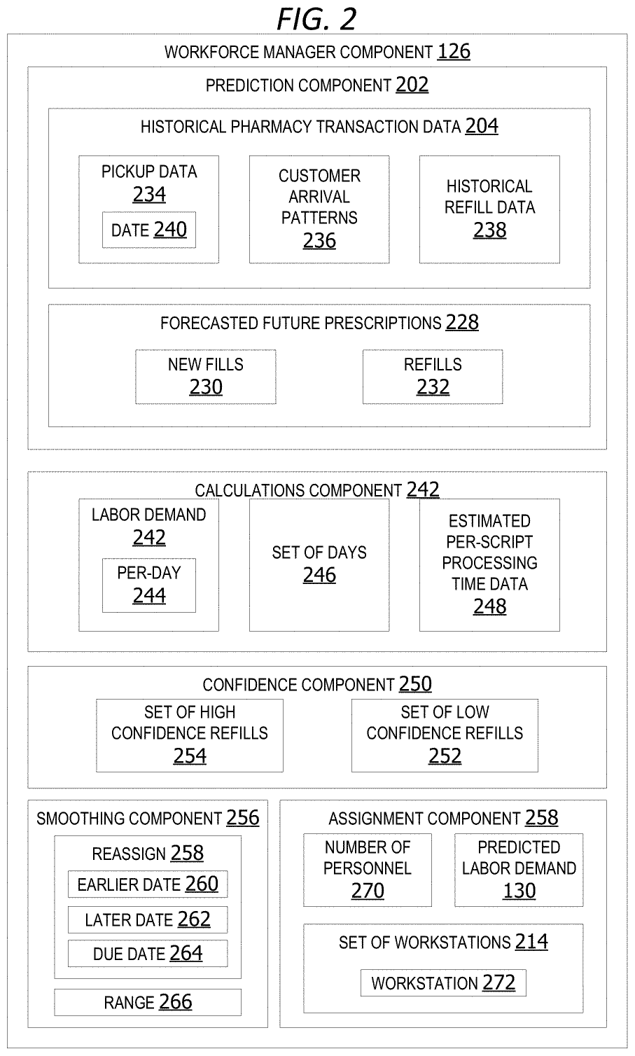

FIG. 2 is an exemplary block diagram illustrating a workforce manager component 126. A prediction component 202 analyzes historical pharmacy transaction data 204 for a selected store or pharmacy. The historical pharmacy transaction data 204 includes data associated with previous/historical new prescriptions and previous prescription refills processed/filled by the selected pharmacy. The historical pharmacy transaction data 204 can include information regarding types of medications, pickup data 234, customer arrival patterns 236, historical refill data 238, dates the prescriptions were received, etc. The pickup data 234 in some examples includes the date 240 on which each new prescription and/or refill prescription was picked up by a customer.

The prediction component 202 utilizes one or more machine learning algorithms to generate forecasted future prescriptions 128 for the selected pharmacy. The forecasted future prescriptions 128 includes both new fills 230 (new prescriptions) predicted to be brought into the pharmacy as well as refills 232 of pre-existing prescriptions predicted to be received by the pharmacy. The forecasted future prescriptions 128 in other examples includes the total number of prescriptions predicted to be handled by the pharmacy on each day in a set of days 246.

The set of days 246 in some examples is a week (7 days) or a business week (5 days). In other examples, the set of days can include several days (half a week), two weeks (fortnight), a month, or any other configurable time-period.

A calculations component 242 calculates labor demand 242 on the per-day 243 level based on the forecasted future prescriptions 128 and estimated per-script processing time data 248. The estimated per-script processing time data 248 includes an estimated amount of time required by pharmacy staff to prepare/process a given prescription (fill the prescription). In other words, the processing time is the amount of time taken to fill a prescription so it is ready for pickup by the customer. The processing time for each prescription varies depending on the type of medication, whether the medication has to be mixed/prepared or is already pre-mixed or pre-prepared, amount of medication needed, dosage, strength of medication, etc.

In other examples, the calculations component 242 calculates raw labor demand at a store-day level based on complexity of each prescription in the forecasted future prescriptions 128 and medication type associated with each forecasted future prescription 128.

A confidence component 250 analyzes item data, including medication data describing each type of medication and customer profile data describing refill pickup dates and customer refill pickup frequency to separate the predicted refill prescriptions into a set of high confidence refills 254 and a set of low confidence refills 252. Thus, in some examples, the confidence component identifies high confidence future refills based on historical refill data and item data associated with each refill prescription in the set of predicted refill prescriptions. A high confidence refill is a refill which has a high probability of being refilled at the selected pharmacy or picked up by the customer on the refill due date 264 or with a refill pickup window. The refill pickup window is a range 266 of days including the refill due date. The refill pickup window can include one or more days before the refill due date and/or one or more days after the refill due date 264.

A smoothing component 256 smooths variable labor demand within the set of days to minimize demand variation across the set of days 246. In other words, the smoothing component 256 attempts to create consistent predicted labor demand across the set of days.

In some examples, the smoothing component 256 can reassign 258 labor demand 242 associated with processing refill prescriptions in the set of high confidence refills to an earlier date 260 or a later date within a range 266 of possible pickup dates associated with a refill due date 264 for each refill prescription in the set of high confidence refills 254. For example, labor demand assigned to a Wednesday can be re-assigned to Tuesday or Thursday if the reassignment smooths the labor demand across two or more of the days. This minimizes labor demand variation across the set of days 246.

An assignment component 268 calculates a number of personnel 270 sufficient to meet the predicted labor demand 130 for each day in the set of days 246. The number of personnel 270 includes the number of pharmacists and/or the number of technicians to assign to each workstation 272 in the set of workstations 274 in the selected pharmacy based on the forecasted future prescriptions for each day and the smoothed labor demand.

An output device 268 in some examples outputs a schedule assigning the calculated number of personnel 270 to each workstation 272 associated with the smoothed labor demand for each day in the set of days. The schedule is a work schedule for the pharmacy during at least one day in the set of days, such as, but not limited to, the schedule 134 in FIG. 1.

In some examples, the workforce manager component considers new-fills and refills data separately to determine the overall future forecast of scripts. The movement of the past data through trend and seasonality is considered to determine the optimal script demand. The special days (Christmas/New year) are been considered separately as these days show special movements/foot traffic. For these days the corresponding values of the last year are multiplied by the corresponding trend component to forecast the current year figures. This method is applied separately for new-fills and refills to determine the overall script demand at a daily level.

In other examples, the workforce manager component determines labor demand based on predicted future prescriptions. New prescriptions can be highly uncertain and difficult to predict with accuracy because the system does not have any information on the customer prior to the new prescription being brought in.

For calculating the total labor hours, in one example, the workforce manager component determines the time taken for catering to a particular prescription. This is an approximate average figure based on the past data. If the time taken to cater to a particular prescription is ‘t’ on an average and the total number of scripts is d, then the total labor hours needed for new prescriptions (new fills) is t*d. For refills, if the time taken to cater to a particular script is ‘t’ on an average and the total number of scripts is d, then the total labor hours needed for refills is calculated as t*d.

The workforce manager component in some examples smooths labor demand. Since the new prescriptions (new fills) are highly uncertain, the total labor hours needed for catering to the script demand remains the same. The refills can also be segregated into two parts, the high confidence refills and the low confidence refills. The refills are associated with some prior information on the customer. The workforce manager component utilizes customer arrival pattern in the past and starts filling the scripts beforehand for high confidence refills only. The confidence score is based on factors like the arrival date of customer (a customer who always come 1-2 days prior/post the actual refill date is a high confidence refill), the type of drug (a highly critical drug is more likely to be picked up) and/or insurance type of customer etc. Such high confidence refills are distributed to previous days so that days with low labor demand and days with high labor demand are more or less balanced.

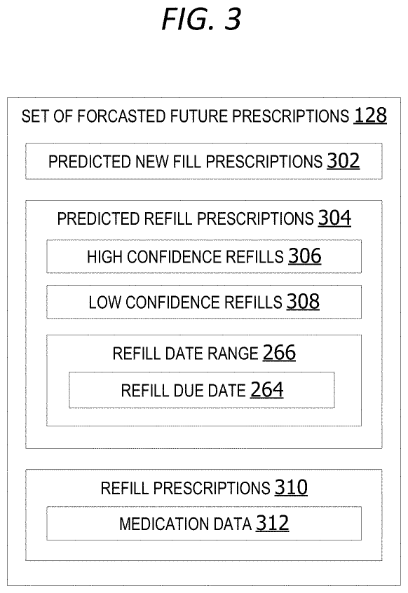

FIG. 3 is an exemplary block diagram illustrating a set of forecasted future prescriptions 128. The set of forecasted future prescriptions 128 includes predicted new fill prescriptions 302 and predicted refill prescriptions 304. The predicted refill prescriptions 304 include high confidence refills 306 having a high probability of being refilled/picked up by a customer and low confidence refills 308 which have a relatively lower likelihood of being refilled/picked up by customers due to the type of medication, customer pickup history, etc.

The medication data 312 for each refill prescription 310 is data describing the type of medication. The medication data 312 can be used to determine whether a prescription is a high confidence or low confidence refill.

For example, if the medication data 312 indicates a refill prescription 310 is for diabetes medication which has been consistently picked up by a customer on time for the last six months if a high confidence refill which is more likely to be picked up than not. Another refill prescription for seasonal allergy medication which has not been picked up consistently by the customer in the past is a low confidence refill.

In some examples, the forecasted future prescriptions 128 includes a refill due date 264 on which refill of the medication is due and a refill date range 266 including a set of days within which the customer is likely to pickup the prescription when it is filled.

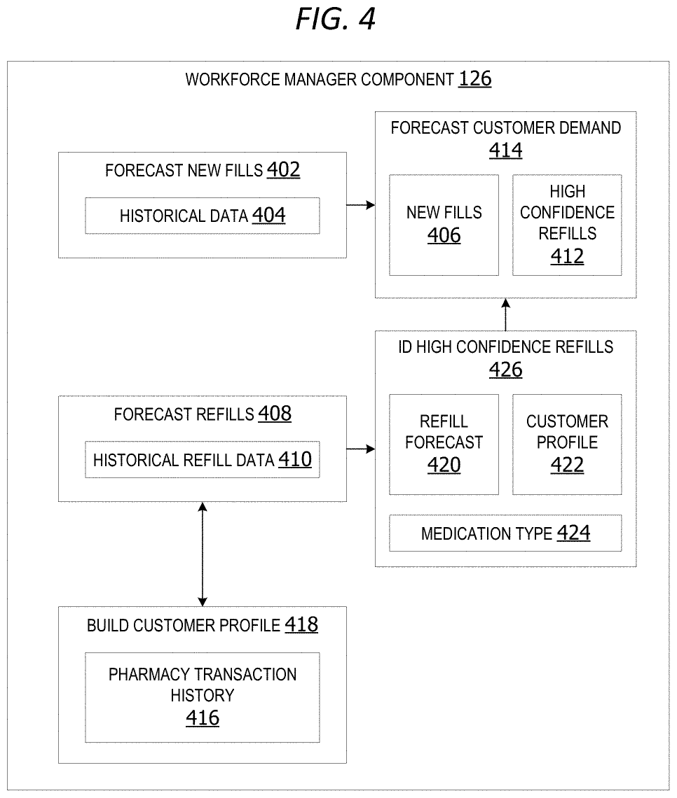

FIG. 4 is an exemplary block diagram illustrating a workforce manager component 126 forecasting customer demand at a per-day level. The workforce manager component 126 can forecast new fills 402 (new prescriptions) based on historical data 404 to generate predicted new fills 406. The workforce manager component 126 can also forecast refills 408 based on analysis of historical refill data 410 to calculate predicted refills 412. The predicted new fills 406 and refills 412 are used to forecast customer demand across a predicted future time-period.

The workforce manager component 126 in other examples builds a customer profile 418 based on pharmacy transaction history 416. The customer profile 422 is anonymized to remove any identifying or personal data form the profiles. The anonymized customer profile 422 in some examples, includes historical pickup dates by the customer for each prescription/medication associated with the customer.

The workforce manager component 126 identifies high confidence refills 426 based on the refill forecast 420, anonymized customer profile 422 and/or medication type 424 of the medication associated with the prescription. The high confidence refills are utilized to smoothen labor demand.

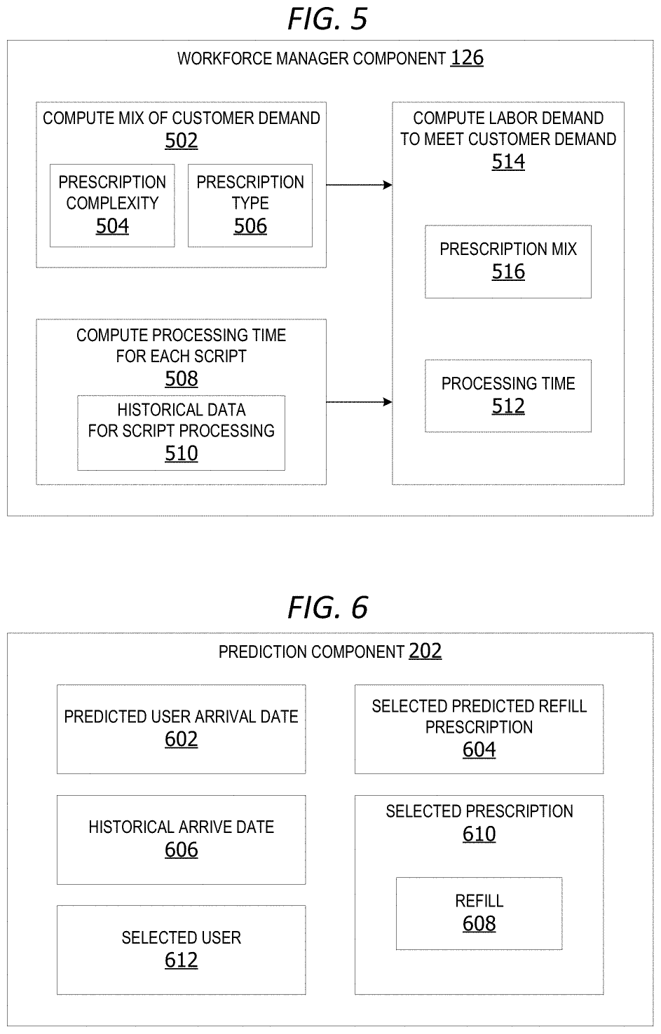

FIG. 5 is an exemplary block diagram illustrating a workforce manager component 126 estimating raw labor demand at a store-day level. The workforce manager component 126 computes mix of customer demand 502 based on prescription complexity 504 and prescription type 506 for each predicted prescription. The workforce manager component 126 computes processing time 512 for each script 508 based on historical data for script processing 510. The workforce manager component 126 computes labor demand to meet customer demand 514 based on the prescription mix 516 and processing time 512 for each prescription.

FIG. 6 is an exemplary block diagram illustrating a prediction component 202 generating predicted pickup dates for forecasted refill prescriptions. The prediction component 202 generates a predicted user arrival date 602 associated with a selected predicted refill prescription 604 based on at least one historical arrival date 606 for at least one refill 608 of a selected prescription 610 by a selected user 612. The predicted prescription refill dates are generated based on the predicted user arrival date 602 for each prescription refill.

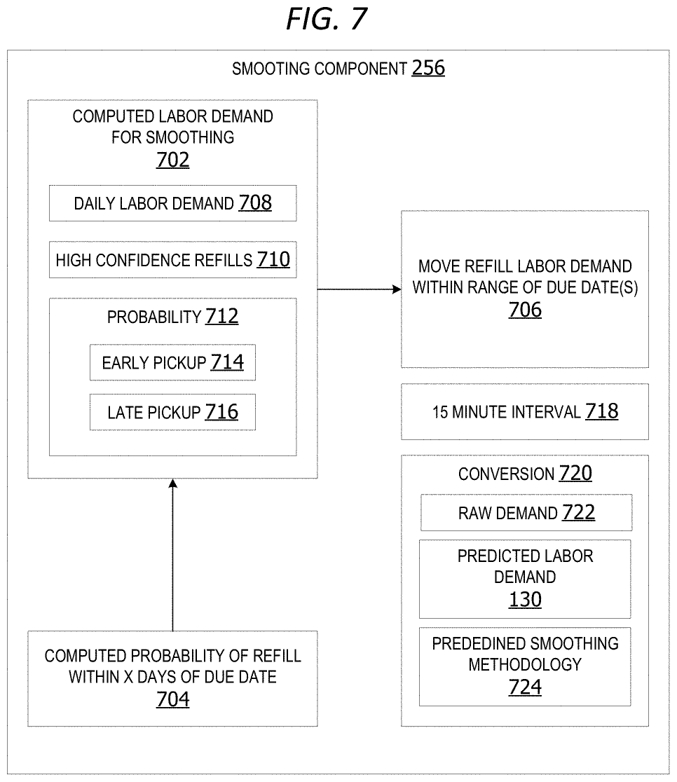

FIG. 7 is an exemplary block diagram illustrating a smoothing component 256 smoothing labor demand across a set of days. The smoothing component 256 utilizes the computed labor demand for smoothing and the probability of refill within “x” days of the prescription refill due date 704 to move refill labor demand within a range of due date(s) associated with the due date. The refill labor demand is moved to minimize variation in daily labor demand and maximize personnel utilization based on the daily labor demand and labor demand for refills, keeping the personnel schedules fixed. In this non-limiting example, the computed labor demand is computed based on daily labor demand 708.

The smoothing component 256 moves the refill labor demand associated with high confidence refills 710 to an earlier date or a later date within the range of due dates to smooth demand within the set of days. In some examples, the range of due dates is calculated based on the probability 712 of early pickup 714 by the customer (user) or late pickup 716 of the refill prescription.

In still other examples, the smoothing component 256 utilizes a fifteen-minute interval 718. The fifteen-minute interval is used during conversion 720 predicted labor demand 130 into raw demand 722 using a predefined smoothing methodology for smoothing demand across each day in the set of days. The smoothing component moves refill labor demand to a date prior to the refill due date to minimize variation in labor demand.

FIG. 8 is an exemplary block diagram illustrating a database 800 storing historical pharmacy transaction data for pharmacy workforce management. In some examples, the schedule 134 generated by the workforce manger component is stored on the database 800. The schedule 134 can include per-workstation scheduling assigning a set of personnel 804 to each workstation 272 during each set of hours for each day 810 in a set of days 246. The set of personnel 804 can include one or more personnel, such as, but not limited to, a pharmacist 806 and/or a technician 808.

In some examples, per-store historical pharmacy transaction data for each pharmacy in a set of one or more pharmacies 816. The per-store historical pharmacy transaction data includes data associated with each pharmacy transaction in a plurality of historical prescription transactions 814 at the selected pharmacy 818. The plurality of historical prescription transactions 814 includes historical transactions associated with new prescriptions and refill prescriptions.

The per-store historical pharmacy transaction data 812 in other examples includes historical prescription refill dates 820. The historical prescription refill dates 820 record the dates on which prescription refills were picked up by users (customers). The prescriptions can be picked up on the refill due date 264, before the refill due date and/or after the refill due date. A refill date range 266 for a given forecast future prescriptions. The range 266 in some examples includes number of days before 822 the refill due date 264 and/or the number of days after 824 the refill due date 264.

The database 800 can optionally store extrinsic data 826. The extrinsic data can include data such as, but not limited to, data associated with seasonality 828, holidays 830, trends 834, geographic data 836, cultural data 832, upcoming events data 838 and/or customer arrival patterns 840. The upcoming events data 838 can include news, weather, parades, sports events, etc. In some example, the prediction component utilizes machine learning to adjust predicted prescription refill dates based on the extrinsic data.

In this example, the database 800 is a single database. In other examples, the database represents a plurality of databases, data structures, files, file systems and/or any other type of data store. The database 800 can also be referred to as a data lake.

FIG. 9 is an exemplary flow chart illustrating operation of the computing device to output a schedule assigning personnel to pharmacy workstations based on predicted labor demand associated with forecasted future prescriptions. The process shown in FIG. 9 is performed by a workforce manager component, executing on a computing device, such as the computing device 102 or the user device 116 in FIG. 1.

The process begins by calculating labor demand for a selected pharmacy on a per-day level at 902. The workforce manager component generates a set of high confidence refills at 904. The workforce manager component is a component such as the workforce manager component 126 in FIG. 1.

The workforce manager component moves labor demand associated with processing high confidence refills within a range of refill dates at 906. The high confidence refill labor demand is moved to smooth labor demand across a set of days.

The workforce manager component determines if the labor demand across the set of days is smooth at 908. If no, the workforce manager component continues moving labor demand associated with high confidence refills to smoothen the demand. When the labor demand is smoothed at 908, the workforce manager component calculates a number of personnel to meet the predicted labor demand at 910. The workforce manager component outputs a schedule assigning the number of personnel to each day at 912. The process terminates thereafter.

While the operations illustrated in FIG. 9 are performed by a computing device, aspects of the disclosure contemplate performance of the operations by other entities. In a non-limiting example, a cloud service performs one or more of the operations.

FIG. 10 is an exemplary flow chart illustrating operation of the computing device to analyze historical data at a per-pharmacy level to perform personnel scheduling based on high confidence refills. The process shown in FIG. 10 is performed by a workforce manager component, executing on a computing device, such as the computing device 102 or the user device 116 in FIG. 1.

The process begins by analyzing historical data at a per-pharmacy level at 1002. The workforce manager component predicts total future prescriptions at a per-day level at 1004. The workforce manager component determines if the labor demand is smooth across the set of days at 1006. If no, the workforce manager component utilizes high confidence refills and predicted pickup window for the high confidence refills to smooth demand at the per-day level at 1008. The workforce manager component schedules personnel at the day level at 1010. The workforce manager component outputs a schedule at 1012. The process terminates thereafter.

While the operations illustrated in FIG. 10 are performed by a computing device, aspects of the disclosure contemplate performance of the operations by other entities. In a non-limiting example, a cloud service performs one or more of the operations.

FIG. 11 is an exemplary flow chart illustrating operation of the computing device to update a workforce schedule based on total forecast prescriptions. The process shown in FIG. 11 is performed by a workforce manager component, executing on a computing device, such as the computing device 102 or the user device 116 in FIG. 1.

The process begins by determining whether to update an existing schedule at 1102. If yes, the workforce manager component forecasts new fills at 1104. The new fills are new prescriptions randomly brought into the pharmacy within a future time-period. The workforce manager component forecasts refills at 1106. The refills are predicted refill prescriptions which are predicted to be picked up by customers during the future time-period. The workforce manager component separates high confidence refills from low confidence refills at 1108. The workforce manager component calculates labor demand per day for a set of days at 1110. The workforce manager component moves high confidence refills across days to smooth demand at 1112. The workforce manager component identifies the number of personnel per workstation based on estimated processing time at 1114. The workforce manager component updates the schedule at 1116 based on the identified number of personnel. The workforce manager component publishes the schedule at 1118. The process terminates thereafter. The schedule in some examples is published as a daily work list specifying which refills to be prepared and on which day.

While the operations illustrated in FIG. 11 are performed by a computing device, aspects of the disclosure contemplate performance of the operations by other entities. In a non-limiting example, a cloud service performs one or more of the operations.

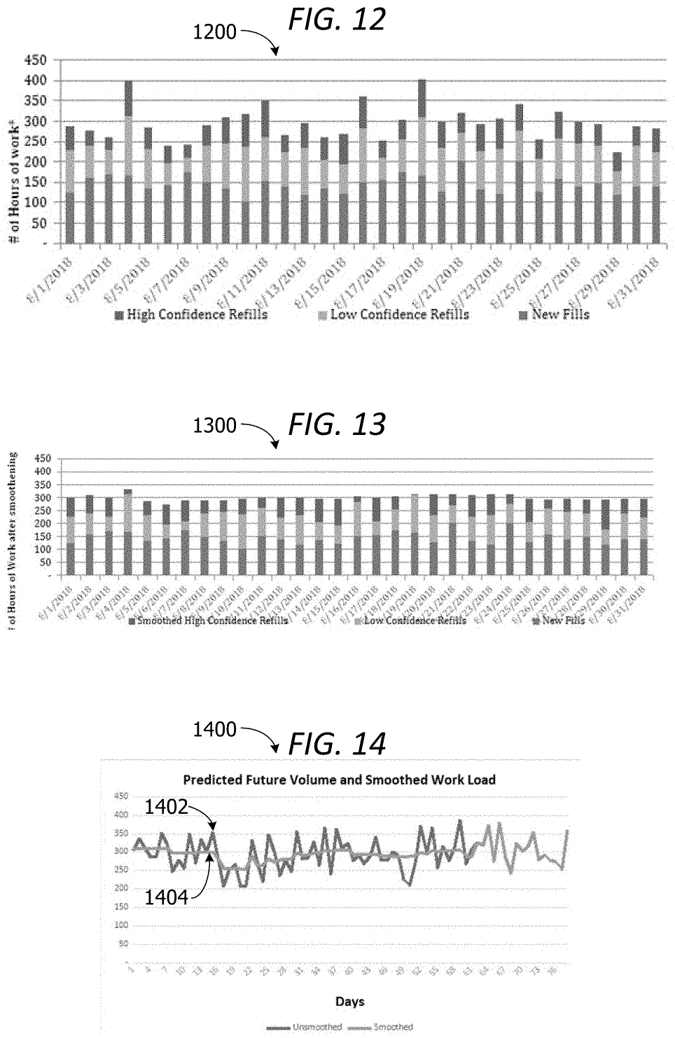

FIG. 12 is an exemplary bar graph 1200 illustrating total predicted labor demand across a set of days. The total predicted labor demand represents labor demand predicted to be sufficient to process the total forecast future prescriptions. The total predicted labor demand is unsmoothed and varies from day-to-day during the represented time-period.

FIG. 13 is an exemplary bar graph 1300 illustrating smoothed labor demand across a set of days. The labor demand has been smoothed using labor demand associated with high confidence refills predicted to occur during the future time-period. The labor demand is smoothed to minimize variations across the set of days.

The system smooths demand to prevent too much work on one day or too little work on another day. The system distributes prescriptions over several days. In one example, if there are 45 high confidence refill prescriptions for a given day, the system assigns 26 prescriptions to the refill due date, fourteen prescriptions to the day after the refill due date and five prescriptions to the day before the due date to prevent idle time.

FIG. 14 is an exemplary line graph 1400 illustrating smoothed labor demand across a set of days. The line 1402 represents unsmoothed labor demand associated with forecasted future prescriptions for a future time-period. The line 1404 represents smoothed labor demand during the future time-period. The blue line 1404 is smoothing out the peaks and valleys so it is more consistent across days. This prevents underutilization or overutilization of resources to improve accuracy of scheduling.

### Additional Examples

In some examples, the system takes into consideration the customer demand and customer arrival patterns to predict efficient labor staffing and optimize labor hours for optimal resource allocation. The system predicts work (prescriptions) coming into a selected pharmacy. The work can include prescription pickup, drop off, etc. Prescription pickup refers to the date on which a customer arrives at a pharmacy to pick up/collect a prescription.

In one non-limiting example, the pharmacy includes five workstations. The system determines number of personnel (pharmacists and technicians) needed at each station. The system uses customer history and drug type to determine confidence level for each predicted prescription refills.

In other examples, the historical pharmacy transaction data is retrieved from a datastore (data lake) where all data is stored. The data store can be implemented as a database Hadoop cluster containing different granularities of data. The system takes data at 15-minute intervals in some non-limiting examples.

A machine learning (ML) platform in some examples performs forecast demand smoothing. Run algorithm in ML platform. The new prescriptions and low confidence refills are classified as uncertain work volume. The high confidence refills are classified as movable within a range of dates (pickup window) of the refill due date. The system moves those high confidence refills within the range to smooth demand. The final output is the number of pharmacists and technicians needed at a store, workstation (pickup station, filling station) for each time interval. The system applies a smoothing algorithm to redistribute labor demand consistently.

In one exemplary scenario, data from data lake is pulled into a machine learning platform where forecasts are generated for all stores at day level. The generated forecasts are converted into fifteen-minute intervals for demand smoothing. The fifteen-minute interval forecasts are converted into raw demand using predefined smoothing standards (raw demand is smoothed using novel methodology). Smoothened demand is fed into the scheduling system to generate personnel/associate hours required at each store/position. The schedule is output into database for labor hour planning at store level.

In some non-limiting examples, the data for all stores/pharmacies is stored in a data lake (database) which is a Hadoop cluster designated for storing prescription-related data. The data granularity is stored at fifteen-minute intervals (15 min. time slots). The data is refreshed on a regular basis. For example, the data can be refreshed once a week, bi-weekly, once a month, bi-monthly, etc.

Data from the lake is pulled into the machine learning platform where forecasts are generated for all stores at day level. Post generating the forecast, the forecast is converted into 15 mins interval for demand smoothing. The fifteen-minute interval forecast are converted into raw demand, using pre-defined standards. The raw demand is smoothened using a smoothing methodology. The smoothened demand is fed into the scheduling system which eventually gives the number of pharmacist and technician hours required at each pharmacy. The final output is a table in a designated database of data lake, which can be accessed by users to plan labor hours at stores/pharmacies.

They system determines optimum allocation of resources through the distribution of labor demand as script demand is not the key factor for determining labor hours. The system takes into consideration all the factors that affect the existing pharmacy/store leading to an accurate forecast. The system considers the allocation of resources by taking into consideration the forecasting estimates made along with the customer arrival patterns leading to a scalable solution. The resources can then be distributed over days leading to an optimal labor demand and saving significant labor cost.

The system considers both new fill and refill demand separately to determine the script demand at the daily level. The system considers the customer arrival patterns and data driven monitoring to improve the forecasting accuracy.

In another exemplary scenario, the system forecasts new prescription fills using the historical time series data and key drivers. The system generates forecast refills using the new prescriptions and past refill information. The workforce manager identifies refills likely to be picked up by customers using the refills forecast and the anonymized customer profile. The workforce manager moves refill labor demand to prior days to minimize variation in daily labor demand and maximize personnel utilization using the daily labor demand and labor demand.

Alternatively, or in addition to the other examples described herein, examples include any combination of the following:
a fifteen-minute interval utilized by the smoothing component for converting predicted labor demand into raw demand using a predefined smoothing methodology for smoothing demand across each day in the set of days;
wherein the schedule further comprises a set of hours assigned to each pharmacist or technician at each workstation within the pharmacy on a per-day level for the selected pharmacy;
data storage device comprising a database storing per-store historical pharmacy transaction data associated with a plurality of historical prescription transactions for each pharmacy in a plurality of pharmacies, wherein the schedule is output to the database for per-hour labor planning at the store level;
the forecast component calculates raw labor demand at a store-day level based on complexity of each prescription in the forecasted future prescriptions and type of medication associated with each forecasted future prescription;
a potential refill time-interval for each high confidence prescription, wherein the potential refill time-interval is calculated based on historical prescription refill dates and a predetermined number of days before a refill due date and a predetermined number of days after the refill due date;
wherein the prescription refill time-interval is a range of days including the refill due date during which a user is predicted to refill a selected prescription;
the smoothing component moves refill labor demand to a date prior to the refill due date to minimize variation in labor demand;
a predicted user arrival date associated with a selected predicted refill prescription, wherein the predicted user arrival date is calculated based on at least one historical arrival date for at least one refill of a selected prescription by a user, wherein the predicted prescription refill dates are generated based on the predicted user arrival date;
extrinsic data, wherein the machine learning component adjusts predicted prescription refill dates based on the extrinsic data, wherein the extrinsic data includes at least one of seasonality, holidays, trends, geographic data, cultural data, weather, upcoming events or customer arrival patterns;
calculating, by a forecast component, labor demand on a per-day level for a selected pharmacy based on a set of forecasted future prescription refills and estimated per-script processing time for each prescription in the set of forecasted future prescriptions;
generating, by a refills confidence component, a set of high confidence refills and a set of low confidence refills based on historical refill data and medication data associated with each refill prescription in the set of predicted refill prescriptions;
moving, by a smoothing component, labor demand associated with processing refill prescriptions in the set of high confidence refills to an earlier date or a later date within a range of possible refill dates associated with a refill due date for each prescription in the set of high confidence refills to smooth labor demand across a set of days;
calculating, by an assignment component, a number of personnel to meet the predicted labor demand for each day in the set of days;
outputting, by a scheduling component, a schedule assigning the calculated number of personnel to each day in the set of days to meet the predicted labor demand;
analyzing, by a machine learning component, per-store historical pharmacy transaction data to generate forecasted future prescriptions, the forecasted future prescriptions comprising a set of predicted refill prescriptions and a set of predicted new prescriptions at a per-day level;
converting the predicted labor demand into raw demand using a fifteen-minute interval and a predefined smoothing methodology for smoothing demand across each day in the set of days;
storing the schedule in a database associated with a data storage device for per-hour labor planning at the store level;
calculating labor demand based on estimated processing time for each prescription in a set of predicted prescription for a given date, wherein the set of predicted prescriptions includes predicted new prescriptions and predicted refill prescriptions;
calculating a potential prescription refill time-interval for each high confidence prescription based on historical prescription refill dates and a predetermined number of days before a refill due date and a predetermined number of days after the refill due date, wherein the prescription refill time-interval is a range of days including the refill due date during which a user is predicted to refill a selected prescription;
adjusting precited prescription refill due dates based on extrinsic data, wherein the extrinsic data includes at least one of seasonality, holidays, trends, geographic data, cultural data, weather, upcoming events or customer arrival patterns;
analyzing per-store historical pharmacy transaction data;
generating forecasted future prescriptions based on the analysis results, the forecasted future prescriptions comprising a set of predicted refill prescriptions and a set of predicted new prescriptions at a per-day level;
calculating labor demand on the per-day level based on the forecasted future prescriptions and estimated per-script processing time data;
creating a set of high confidence refills and a set of low confidence refills based on historical refill data and medication data associated with each refill prescription in the set of predicted refill prescriptions;
smoothing labor demand across a set of days by moving labor demand associated with processing at least one refill prescription in the set of high confidence refills to an earlier date or a later date within a range of predicted refill dates for each high confidence refill;
outputting a number of personnel associated with the smoothed labor demand for utilization in creating a workforce schedule for each day in the set of days;
wherein the workforce manager component, when further executed by a computer, cause the computer to perform operations comprising converting the predicted labor demand into raw demand using a fifteen-minute interval and a predefined smoothing methodology for smoothing demand across each day in the set of days;
wherein the workforce manager component, when further executed by a computer, cause the computer to perform operations comprising calculating a potential prescription refill time-interval for each high confidence prescription based on historical prescription refill dates and a predetermined number of days before a refill due date and a predetermined number of days after the refill due date;
wherein the prescription refill time-interval is a range of days including the refill due date during which a user is predicted to refill a selected prescription; and
wherein the workforce manager component, when further executed by a computer, cause the computer to perform operations comprising adjusting precited prescription refill due dates based on extrinsic data, wherein the extrinsic data includes at least one of seasonality, holidays, trends, geographic data, cultural data, weather, upcoming events or customer arrival patterns.

At least a portion of the functionality of the various elements in FIG. 1, FIG. 2, FIG. 3, FIG. 4, FIG. 5, FIG. 6, FIG. 7, FIG. 8 and FIG. 9 can be performed by other elements in FIG. 1, FIG. 2, FIG. 3, FIG. 4, FIG. 5, FIG. 6, FIG. 7, FIG. 8 and FIG. 9, or an entity (e.g., processor 106, web service, server, application program, computing device, etc.) not shown in FIG. 1, FIG. 2, FIG. 3, FIG. 4, FIG. 5, FIG. 6, FIG. 7, FIG. 8 and FIG. 9.

In some examples, the operations illustrated in FIG. 9, FIG. 10 and FIG. 11 can be implemented as software instructions encoded on a computer-readable medium, in hardware programmed or designed to perform the operations, or both. For example, aspects of the disclosure can be implemented as a system on a chip or other circuitry including a plurality of interconnected, electrically conductive elements.

In other examples, a computer readable medium having instructions recorded thereon which when executed by a computer device cause the computer device to cooperate in performing a method of workforce management using high confidence forecast refill prescriptions, the method comprising calculating labor demand on a per-day level for a selected pharmacy based on a set of forecasted future prescription refills and estimated per-script processing time for each prescription in the set of forecasted future prescriptions; generating a set of high confidence refills and a set of low confidence refills based on historical refill data and medication data associated with each refill prescription in the set of predicted refill prescriptions; moving labor demand associated with processing refill prescriptions in the set of high confidence refills to an earlier date or a later date within a range of possible refill dates associated with a refill due date for each prescription in the set of high confidence refills to smooth labor demand across a set of days; and calculating a number of personnel to meet the predicted labor demand for each day in the set of days; and outputting a schedule assigning the calculated number of personnel to each day in the set of days to meet the predicted labor demand.

While the aspects of the disclosure have been described in terms of various examples with their associated operations, a person skilled in the art would appreciate that a combination of operations from any number of different examples is also within scope of the aspects of the disclosure.

The term “Wi-Fi” as used herein refers, in some examples, to a wireless local area network using high frequency radio signals for the transmission of data. The term “BLUETOOTH®” as used herein refers, in some examples, to a wireless technology standard for exchanging data over short distances using short wavelength radio transmission. The term “NFC” as used herein refers, in some examples, to a short-range high frequency wireless communication technology for the exchange of data over short distances.

While no personally identifiable information is tracked by aspects of the disclosure, examples have been described with reference to prescription-related data monitored and/or collected from one or more pharmacies. In some examples, notice is provided to the users of the collection of the data (e.g., via a dialog box or preference setting) and users are given the opportunity to give or deny consent for the monitoring and/or collection. The consent can take the form of opt-in consent or opt-out consent.

### Exemplary Operating Environment

Exemplary computer-readable media include flash memory drives, digital versatile discs (DVDs), compact discs (CDs), floppy disks, and tape cassettes.

By way of example and not limitation, computer-readable media comprise computer storage media and communication media. Computer storage media include volatile and nonvolatile, removable and non-removable media implemented in any method or technology for storage of information such as computer-readable instructions, data structures, program modules and the like. Computer storage media are tangible and mutually exclusive to communication media. Computer storage media are implemented in hardware and exclude carrier waves and propagated signals. Computer storage media for purposes of this disclosure are not signals per se. Exemplary computer storage media include hard disks, flash drives, and other solid-state memory. In contrast, communication media typically embody computer-readable instructions, data structures, program modules, or the like, in a modulated data signal such as a carrier wave or other transport mechanism and include any information delivery media.

Although described in connection with an exemplary computing system environment, examples of the disclosure are capable of implementation with numerous other general purpose or special purpose computing system environments, configurations, or devices.

Examples of well-known computing systems, environments, and/or configurations that can be suitable for use with aspects of the disclosure include, but are not limited to, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. Such systems or devices can accept input from the user in any way, including from input devices such as a keyboard or pointing device, via gesture input, proximity input (such as by hovering), and/or via voice input.

Examples of the disclosure can be described in the general context of computer-executable instructions, such as program modules, executed by one or more computers or other devices in software, firmware, hardware, or a combination thereof. The computer-executable instructions can be organized into one or more computer-executable components or modules. Generally, program modules include, but are not limited to, routines, programs, objects, components, and data structures that perform tasks or implement abstract data types. Aspects of the disclosure can be implemented with any number and organization of such components or modules. For example, aspects of the disclosure are not limited to the specific computer-executable instructions or the specific components or modules illustrated in the figures and described herein. Other examples of the disclosure can include different computer-executable instructions or components having more functionality or less functionality than illustrated and described herein.

In examples involving a general-purpose computer, aspects of the disclosure transform the general-purpose computer into a special-purpose computing device when configured to execute the instructions described herein.

The examples illustrated and described herein as well as examples not specifically described herein but within the scope of aspects of the disclosure constitute exemplary means for pharmacy workforce management based on forecast refills and new fill prescriptions. For example, the elements illustrated in FIG. 1, FIG. 2, FIG. 3, FIG. 4, FIG. 5, FIG. 6, FIG. 7, FIG. 8 and FIG. 9, such as when encoded to perform the operations illustrated in FIG. 9, FIG. 10 and FIG. 11, constitute exemplary means for calculating labor demand on a per-day level for a selected pharmacy based on a set of forecasted future prescription refills and estimated per-script processing time for each prescription in the set of forecasted future prescriptions; generating a set of high confidence refills and a set of low confidence refills based on historical refill data and medication data associated with each refill prescription in the set of predicted refill prescriptions; moving labor demand associated with processing refill prescriptions in the set of high confidence refills to an earlier date or a later date within a range of possible refill dates associated with a refill due date for each prescription in the set of high confidence refills to smooth labor demand across a set of days; calculating a number of personnel to meet the predicted labor demand for each day in the set of days; and outputting a schedule assigning the calculated number of personnel to each day in the set of days to meet the predicted labor demand.

Other non-limiting examples provide one or more computer storage devices having a first computer-executable instructions stored thereon for providing pharmacy workforce management. When executed by a computer, the computer performs operations including analyzing per-store historical pharmacy transaction data; generating forecasted future prescriptions based on the analysis results, the forecasted future prescriptions comprising a set of predicted refill prescriptions and a set of predicted new prescriptions at a per-day level; calculating labor demand on the per-day level based on the forecasted future prescriptions and estimated per-script processing time data; creating a set of high confidence refills and a set of low confidence refills based on historical refill data and medication data associated with each refill prescription in the set of predicted refill prescriptions; smoothing labor demand across a set of days by moving labor demand associated with processing at least one refill prescription in the set of high confidence refills to an earlier date or a later date within a range of predicted refill dates for each high confidence refill; and outputting a number of personnel associated with the smoothed labor demand for utilization in creating a workforce schedule for each day in the set of days.

The order of execution or performance of the operations in examples of the disclosure illustrated and described herein is not essential, unless otherwise specified. That is, the operations can be performed in any order, unless otherwise specified, and examples of the disclosure can include additional or fewer operations than those disclosed herein. For example, it is contemplated that executing or performing an operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there can be additional elements other than the listed elements. The term “exemplary” is intended to mean “an example of” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A system for performing unified pharmacy workforce management, the system comprising:
at least one processor communicatively coupled to a memory;
a prediction component, implemented on the at least one processor, analyzes per-store historical pharmacy transaction data to generate forecasted future prescriptions, the forecasted future prescriptions comprising a set of predicted refill prescriptions and a set of predicted new prescriptions at a per-day level;
a calculations component, implemented on the at least one processor, calculates labor demand on the per-day level based on the forecasted future prescriptions and estimated per-script processing time data;
a confidence component, implemented on the at least one processor, separates the set of predicted refill prescriptions into a set of high confidence refills and a set of low confidence refills based on historical refill data and item data associated with each refill prescription in the set of predicted refill prescriptions;
a smoothing component, implemented on the at least one processor, reassigns the labor demand associated with processing each refill prescription in the set of high confidence refills to an earlier date or a later date within a range of possible pickup dates associated with a refill due date for each refill prescription in the set of high confidence refills to smooth the labor demand variation across a set of days;
an assignment component, implemented by the at least one processor, calculates a number of personnel sufficient to meet the labor demand predicted for each day in the set of days; and
an output device outputs a schedule assigning the calculated number of personnel to each workstation associated with the smoothed labor demand for each day in the set of days.

2. The system of claim 1, further comprising:
a fifteen-minute interval utilized by the smoothing component for converting predicted labor demand into raw demand using a predefined smoothing methodology for smoothing demand across each day in the set of days.

3. The system of claim 1, wherein the schedule further comprises a set of hours assigned to each pharmacist or technician at each workstation within a pharmacy on the per-day level for the pharmacy.

4. The system of claim 1, further comprising:
a data storage device comprising a database storing per-store historical pharmacy transaction data associated with a plurality of historical prescription transactions for each pharmacy in a plurality of pharmacies, wherein the schedule is output to the database for per-hour labor planning at a store level.

5. The system of claim 1, further comprising:
the calculations component calculates raw labor demand at a store-day level based on complexity of each prescription in the forecasted future prescriptions and medication type associated with each forecasted future prescription.

6. The system of claim 1, further comprising:
a refill date range for each high confidence prescription, wherein the refill date range comprises a range of days for potential prescription pickup calculated based on historical prescription refill dates, the refill date range comprising a number of days before the refill due date and a number of days after the refill due date during which a user is predicted to refill a selected prescription.

7. The system of claim 1, further comprising:
the smoothing component moves refill labor demand to a date prior to the refill due date to minimize variation in the labor demand.

8. The system of claim 1, further comprising:
a predicted user arrival date associated with a selected predicted refill prescription, wherein the predicted user arrival date is calculated based on at least one historical arrival date for at least one refill of a selected prescription by a user, wherein the predicted prescription refill dates are generated based on the predicted user arrival date.

9. The system of claim 1, further comprising:
extrinsic data, wherein the prediction component utilizes machine learning to adjust predicted prescription refill dates based on the extrinsic data, wherein the extrinsic data includes at least one of seasonality, holidays, trends, geographic data, cultural data, weather, upcoming events or customer arrival patterns.

10. A computer-implemented method for performing unified pharmacy workforce management, the method comprising:
calculating, by a forecast component, labor demand on a per-day level for a selected pharmacy based on a set of forecasted future prescription refills and estimated per-script processing time for each prescription in the set of forecasted future prescriptions;
generating, by a refills confidence component, a set of high confidence refills and a set of low confidence refills based on historical refill data and medication data associated with each refill prescription in the set of predicted refill prescriptions;
moving, by a smoothing component, the labor demand associated with processing each refill prescription in the set of high confidence refills to an earlier date or a later date within a range of possible refill dates associated with a refill due date for each prescription in the set of high confidence refills to smooth the labor demand across a set of days;
calculating, by an assignment component, a number of personnel to meet the labor demand predicted for each day in the set of days; and
outputting, by a scheduling component, a schedule assigning the calculated number of personnel to each day in the set of days based on the smoothed labor demand.

11. The computer-implemented method of claim 10, further comprising:
analyzing, by a prediction component, per-store historical pharmacy transaction data to generate the set of forecasted future prescriptions, the set of forecasted future prescriptions comprising a set of predicted refill prescriptions and a set of predicted new prescriptions at the per-day level.

12. The computer-implemented method of claim 10, further comprising:
converting predicted labor demand into raw demand using a fifteen-minute interval and a predefined smoothing methodology for smoothing demand across each day in the set of days.

13. The computer-implemented method of claim 10, further comprising:
storing the schedule in a database associated with a data storage device for per-hour labor planning at a store level.

14. The computer-implemented method of claim 10, further comprising:
calculating the labor demand based on estimated processing time for each prescription in a set of predicted prescription for a given date, wherein the set of predicted prescriptions includes predicted new prescriptions and predicted refill prescriptions.

15. The computer-implemented method of claim 10, further comprising:
calculating a prescription refill time-interval for each high confidence prescription based on historical prescription refill dates and a predetermined number of days before the refill due date and a predetermined number of days after the refill due date, wherein the prescription refill time-interval is a range of days including the refill due date during which a user is predicted to refill a selected prescription.

16. The computer-implemented method of claim 10, further comprising:
adjusting predicted prescription refill due dates based on extrinsic data, wherein the extrinsic data includes at least one of seasonality, holidays, trends, geographic data, cultural data, weather, upcoming events or customer arrival patterns.

17. One or more computer storage devices, having computer-executable instructions for performing unified pharmacy workforce management by a workforce manager component, that, when executed by a computer cause the computer to perform operations comprising:
analyzing per-store historical pharmacy transaction data;
generating forecasted future prescriptions based on analysis results, the forecasted future prescriptions comprising a set of predicted refill prescriptions and a set of predicted new prescriptions at a per-day level;
calculating labor demand on the per-day level based on the forecasted future prescriptions and estimated per-script processing time data;
creating a set of high confidence refills and a set of low confidence refills based on historical refill data and medication data associated with each refill prescription in the set of predicted refill prescriptions;
smoothing the labor demand across a set of days by moving the labor demand associated with processing at least one refill prescription in the set of high confidence refills to an earlier date or a later date within a range of predicted refill dates for each high confidence refill; and
outputting a number of personnel associated with the smoothed labor demand for utilization in creating a workforce schedule for each day in the set of days.

18. The one or more computer storage devices of claim 17, wherein the workforce manager component, when further executed by a computer, cause the computer to perform operations comprising:
converting predicted labor demand into raw demand using a fifteen-minute interval and a predefined smoothing methodology for smoothing demand across each day in the set of days.

19. The one or more computer storage devices of claim 17, wherein the workforce manager component, when further executed by a computer, cause the computer to perform operations comprising:
calculating a prescription refill time-interval for each high confidence prescription based on historical prescription refill dates and a predetermined number of days before a refill due date and a predetermined number of days after the refill due date, wherein the prescription refill time-interval is a range of days including the refill due date during which a user is predicted to refill a selected prescription.

20. The one or more computer storage devices of claim 17, wherein the workforce manager component, when further executed by a computer, cause the computer to perform operations comprising:
adjusting precited prescription refill due dates based on extrinsic data, wherein the extrinsic data includes at least one of seasonality, holidays, trends, geographic data, cultural data, weather, upcoming events or customer arrival patterns.
